(19) United States
(12) Patent Application Publication
(10) Pub. No.: US 2024/0335274 A1
(43) Pub. Date: Oct. 10, 2024

(54) URETHRAL STENT AND STENT DEPLOYMENT SYSTEM FOR TREATING BPH AND OTHER VASCULATURE

(71) Applicants: Show-Mean Steve Wu, Fremont, CA (US); Brandon Le, Lake Forest, CA (US)

(72) Inventors: Show-Mean Steve Wu, Fremont, CA (US); Brandon Le, Lake Forest, CA (US)

(21) Appl. No.: 18/614,519

(22) Filed: Mar. 22, 2024

Related U.S. Application Data
(60) Provisional application No. 63/457,438, filed on Apr. 6, 2023.

(51) Int. Cl. A61F 2/04; A61F 2/00; A61F 2/966

(52) U.S. Cl. CPC A61F 2/04; A61F 2002/0081; A61F 2002/047; A61F 2002/9665; A61F 2250/001; A61F 2250/0039

(57) ABSTRACT

A stent (10) to be positioned within a urethra (611U) of a patient (611) includes a first stent end (12), an opposed second stent end (14), and a stent body (16) that extends between the first stent end (12) and the second stent end (14). Each of the stent ends (12, 14) is flared relative to the stent body (16). The stent body (16) has one or more bumps (18) from the first stent end (12) to the second stent end (14). The stent (10) moves from a compressed configuration (10C) as the stent (10) is initially positioned within the urethra (611U), to an expanded configuration (10E) as the stent (10) is deployed at a desired location within the urethra (611U). A stent deployment system (220) that positions the stent (10) within the urethra (611U) includes an inner shaft (224), at least one tube ring (226) positioned about the inner shaft (224), and an outer sheath (228) that is configured to substantially encircle the stent (10) as mounted about the inner shaft (224) and the at least one tube ring (226).

### RELATED APPLICATION

This application claims priority on U.S. Provisional Application Ser. No. 63/457,438, filed on Apr. 6, 2023, and entitled “URETHRAL STENT FOR TREATING BPH AND OTHER VASCULATURE”. As far as permitted, the contents of U.S. Provisional application Ser. No. 63/457,438 are incorporated in their entirety herein by reference.

### BACKGROUND

The prostate is a walnut-shaped gland that wraps around the urethra through which urine is expelled from the bladder. The prostate also plays a crucial role in the reproductive system of men. Although the prostate gland starts out small, it tends to enlarge as a man ages. An excessively enlarged prostate results in a disease known as benign prostatic hyperplasia (BPH).

BPH is one of the most common medical conditions that affect men, especially older men. It has been reported that, in the United States, more than half of all men have histopathologic evidence of BPH by age 60, and, by age 85, approximately 9 out of 10 men suffer from the condition. Although BPH is rarely life threatening, it can adversely impact proper functioning of the urethra and can lead to numerous clinical conditions including urinary retention, renal insufficiency, recurrent urinary tract infection, incontinence, hematuria, and bladder stones.

Many treatments have been used to treat BPH. For example, surgical procedures for treating BPH symptoms include Transurethral Resection of Prostate (TURP), Transurethral Electrovaporization of Prostate (TVP), Transurethral Incision of the Prostate (TUIP), Laser Prostatectomy and Open Prostatectomy. Minimally invasive procedures for treating BPH symptoms include Transurethral Microwave Thermotherapy (TUMT), Transurethral Needle Ablation (TUNA), Interstitial Laser Coagulation (ILC), and Prostatic (Urethral) Stents.

Despite the various options noted, there is a continuing desire to develop additional procedures, especially minimally invasive procedures, that can be utilized to effectively treat BPH symptoms.

### SUMMARY

The present invention is directed toward a stent that is configured to be positioned within a urethra of a patient. In various embodiments, the stent includes a first stent end, an opposed second stent end, and a stent body that extends between the first stent end and the second stent end. At least one of the first stent end and the second stent end is flared relative to the stent body.

In some embodiments, each of the first stent end and the second stent end is flared relative to the stent body.

In certain embodiments, the first stent end has a first end diameter, the stent body has a body diameter; and a ratio of the first end diameter of the first stent end to the body diameter of the stent body is between approximately 1.05:1 and 1.50:1.

In some embodiments, the second stent end has a second end diameter, and a ratio of the second end diameter of the second stent end to the body diameter of the stent body is between approximately 1.05:1 and 1.50:1.

In certain embodiments, the stent body has one or more bumps along the stent body from the first stent end to the second stent end.

In some embodiments, the stent body has a plurality of bumps along the stent body from the first stent end to the second stent end.

In many embodiments, the stent body is substantially tubular-shaped.

In various embodiments, the stent is configured to move from a compressed configuration as the stent is initially positioned within the urethra of the patient, to an expanded configuration as the stent is deployed at a desired location within the urethra of the patient.

In some embodiments, the stent is a self-expanding stent that is biased to spontaneously move from the compressed configuration to the expanded configuration.

The present invention is further directed toward a stent assembly including the stent as described above; and a stent deployment system that is configured to position the stent within the urethra of the patient.

In many embodiments, the stent deployment system includes an inner shaft, at least one tube ring positioned about the inner shaft, and an outer sheath that is configured to substantially encircle the inner shaft and the at least one tube ring, the outer sheath defining an inner cavity therewithin.

In certain embodiments, the stent is mounted about the inner shaft and the at least one tube ring prior to being positioned within the urethra of the patient so that the stent is maintained in a compressed configuration.

In some embodiments, the stent as mounted about the inner shaft and the at least one tube ring is positioned within the inner cavity of the outer sheath prior to being positioned within the urethra of the patient.

The present invention is also directed toward a stent assembly including a stent including a first stent end, an opposed second stent end, and a stent body that extends between the first stent end and the second stent end; and a stent deployment system that is configured to position the stent within a urethra of a patient, the stent deployment system including an inner shaft, at least one tube ring positioned about the inner shaft, and an outer sheath that is configured to substantially encircle the inner shaft and the at least one tube ring, the outer sheath defining an inner cavity therewithin; wherein the stent is positioned within the inner cavity of the outer sheath as the stent is being positioned within the urethra of the patient.

In certain embodiments, the stent assembly further includes a crimping head that is configured to crimp the stent onto the inner shaft and the at least one tube ring prior to being positioned within the urethra of the patient so that the stent is maintained in the compressed configuration.

In certain implementations, the present invention is further directed toward a stent assembly including a stent including a first stent end, an opposed second stent end, and a stent body that extends between the first stent end and the second stent end, the stent body having one or more bumps along the stent body from the first stent end to the second stent end, each of the first stent end and the second stent end being flared relative to the stent body; and a stent deployment system that is configured to position the stent within a urethra of a patient, the stent deployment system including an inner shaft, at least one tube ring positioned about the inner shaft, and an outer sheath that is configured to substantially encircle the inner shaft and the at least one tube ring, the outer sheath defining an inner cavity therewithin; wherein the stent is mounted about the inner shaft and the at least one tube ring prior to being positioned within the urethra of the patient so that the stent is maintained in a compressed configuration; wherein the stent as mounted about the inner shaft and the at least one tube ring is positioned within the inner cavity of the outer sheath prior to being positioned within the urethra of the patient so that the stent is further maintained in the compressed configuration; wherein the first stent end has a first end diameter, the second stent end has a second end diameter, and the stent body has a body diameter; wherein a ratio of the first end diameter of the first stent end to the body diameter of the stent body is between approximately 1.05:1 and 1.50:1; wherein a ratio of the second end diameter of the second stent end to the body diameter of the stent body is between approximately 1.05:1 and 1.50:1; wherein the stent is configured to move from the compressed configuration as the stent is initially positioned within the urethra of the patient, to an expanded configuration as the stent is deployed at a desired location within the urethra of the patient; and wherein the stent is a self-expanding stent that is biased to spontaneously move from the compressed configuration to the expanded configuration when the stent is removed from within the inner cavity of the outer sheath.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1A is a simplified schematic perspective view illustration of an embodiment of a urethral stent having features of the present invention;

FIG. 1B is a simplified schematic side view illustration of the urethral stent illustrated in FIG. 1A, the urethral stent being shown in a compressed configuration;

FIG. 1C is a simplified schematic side view illustration of the urethral stent illustrated in FIG. 1A, the urethral stent being shown in an expanded configuration;

FIG. 2 is a simplified schematic side view illustration of an embodiment of the urethral stent, and an embodiment of a stent deployment system having features of the present invention that is usable for positioning the urethral stent within a urethra of a patient;

FIG. 3 is a flowchart that illustrates a method for utilizing the stent deployment system of FIG. 2 to position the urethral stent at a desired location within the urethra of the patient;

FIG. 4 is a simplified schematic side view illustration of the urethral stent being mounted (crimped) onto a portion of the stent deployment system so that the urethral stent is in the compressed configuration;

FIG. 5 is a simplified schematic side view illustration of the crimped urethral stent being positioned within an outer sheath of the stent deployment system so that the urethral stent remains in the compressed configuration;

FIG. 6 is a simplified schematic side view illustration of the urethral stent and the stent deployment system of FIG. 2 during initial deployment of the urethral stent toward the desired location within the urethra of the patient;

FIG. 7 is a simplified schematic side view illustration of the urethral stent and the stent deployment system of FIG. 2 as the urethral stent and the stent deployment system have arrived at least approximately at the desired location within the urethra of the patient;

FIG. 8 is a simplified schematic side view illustration of the urethral stent and the stent deployment system of FIG. 2 after partial withdrawal of the outer sheath relative to the urethral stent, with the urethral stent now having moved partially toward the expanded configuration and being partially positioned at the desired location within the urethra of the patient;

FIG. 9 is a simplified schematic side view illustration of the urethral stent and a portion of the stent deployment system of FIG. 2 after full withdrawal of the outer sheath relative to the urethral stent, with the urethral stent now fully in the expanded configuration and being fixed in position at the desired location within the urethra of the patient; and

FIG. 10 is a simplified schematic side view illustration of the urethral stent after full withdrawal of the stent deployment system relative to the urethral stent, with the urethral stent now fully in the expanded configuration and being fixed in position at the desired location within the urethra of the patient.

While embodiments of the present invention are susceptible to various modifications and alternative forms, specifics thereof have been shown by way of example and drawings, and are described in detail herein. It is understood, however, that the scope herein is not limited to the particular embodiments described. On the contrary, the intention is to cover modifications, equivalents, and alternatives falling within the spirit and scope herein.

### DESCRIPTION

Embodiments of the present invention are described herein in the context of a urethral stent (also sometimes referred to as a prostatic stent), and a stent deployment system and related method for deployment of the urethral stent within a urethra of a patient. The urethral stent and the stent deployment system utilized to deploy the urethral stent within the urethra of the patient are sometimes referred to collectively herein as a “stent assembly”. In various implementations, the stent assembly is usable in a minimally invasive procedure for treating BPH symptoms, such as urinary outflow obstruction symptoms, and other vasculature. More particularly, through use of the stent assembly of the present invention, the region of the urethra that is surrounded by the prostate is mechanically supported in order to reduce constriction of the urethra that can be caused by an enlarged prostate.

Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings. The same or similar reference indicators will be used throughout the drawings and the following detailed description to refer to the same or like parts.

In the interest of clarity, not all of the routine features of the implementations described herein are shown and described. It will, of course, be appreciated that in the development of any such actual implementations, numerous implementation-specific decisions must be made in order to achieve the developer's specific goals, such as compliance with application-related and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

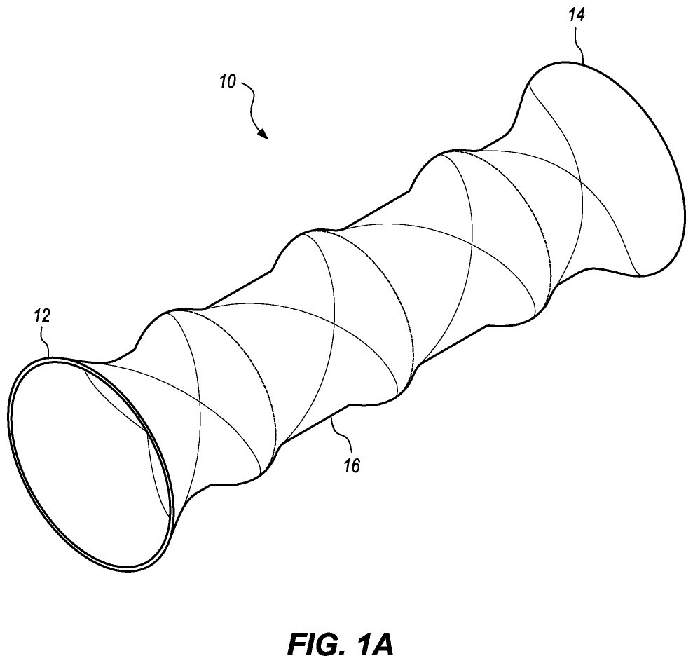

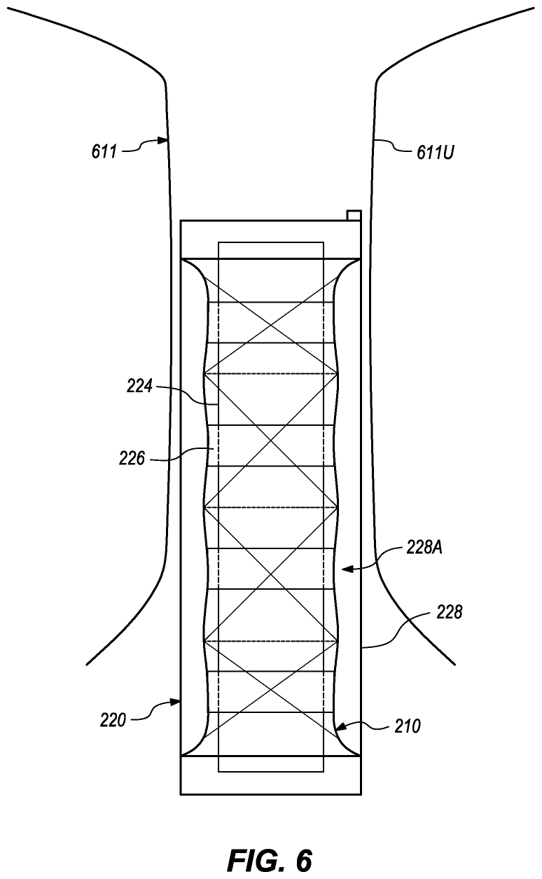

FIG. 1A is a simplified schematic perspective view illustration of an embodiment of a urethral stent 10 (also sometimes referred to as a “stent”) having features of the present invention. In particular, as described in detail herein, the urethral stent 10 is usable in a minimally invasive procedure for delivery and/or deployment within a urethra 611U (illustrated in FIG. 6) of a patient 611 (illustrated in FIG. 6) for treating BPH symptoms, such as urinary outflow obstruction symptoms, and other vasculature.

As described herein, the urethral stent 10, or prostatic stent, is a flexible device that can be expanded after being inserted, positioned and/or deployed within the urethra 611U of the patient 611. The urethral stent 10 is configured to mechanically support the urethra 611U by pushing any obstructing prostatic tissue away from the urethra 611U. This reduces any constriction of the urethra 611U and improves urine flow past the prostate gland thereby reducing the severity of BPH symptoms.

The design of the urethral stent 10 can be varied to suit the requirements of the patient 611 in which the urethral stent 10 is being deployed. In various embodiments, the urethral stent 10 can be substantially tubular-shaped and can be appropriately sized for deployment within the urethra 611U of the patient 611. As illustrated in the embodiment shown in FIG. 1A, the urethral stent 10 can include a first stent end 12, an opposed second stent end 14, and a stent body 16 that is integrally formed with and extends between the first stent end 12 and the second stent end 14. Alternatively, the urethral stent 10 can have another suitable design. For example, in certain alternative embodiments, the urethral stent 10 can include more components than those specifically illustrated and described in relation to FIG. 1A.

It is appreciated that the reference to a “first stent end” and a “second stent end” is merely for convenience and ease of discussion, and either stent end 12, 14 can be referred to as the “first stent end” or the “second stent end”.

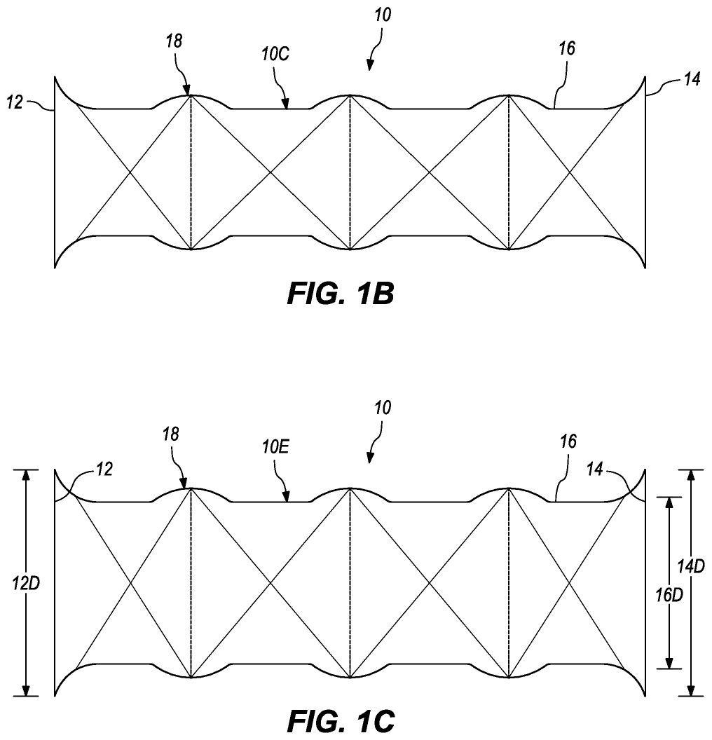

In various embodiments, as described in greater detail herein below, the urethral stent 10 is configured to be positioned and expanded within the urethra 611U of the patient 611. More particularly, in many embodiments, the urethral stent 10 has a lattice-type structure, formed from flexible and/or expandable materials, which is configured to move from a compressed configuration 10C (illustrated in FIG. 1B), as the urethral stent 10 is initially being positioned and/or deployed toward and within the urethra 611U, to an expanded configuration 10E (illustrated in FIG. 1C), as the urethral stent 10 is being accurately positioned and/or deployed at a desired location within the urethra 611U of the patient 611. In some embodiments, the urethral stent 10 can be a self-expanding stent that is biased to spontaneously move from the compressed configuration 10C to the expanded configuration 10E when any external forces and/or structures are removed, which otherwise serve to maintain the urethral stent 10 in the compressed configuration 10C. For example, in various embodiments, the urethral stent 10 is positioned and/or deployed within the urethra 611U of the patient 611 through use of a stent deployment system 220 (illustrated in FIG. 2) that is configured to maintain the urethral stent 10 in the compressed configuration 10C until the urethral stent 10 is accurately deployed at the desired location within the urethra 611U of the patient 611. Alternatively, in other embodiments, the urethral stent 10 can have a different design and/or can employ a different means for moving the urethral stent 10 from the compressed configuration 10C to the expanded configuration 10E.

In certain embodiments, when the urethral stent 10 moves from the compressed configuration 10C to the expanded configuration 10E, the urethral stent 10 expands only in circumference and/or diameter, and a longitudinal length of the urethral stent 10 does not expand. In other embodiments, when the urethral stent 10 moves from the compressed configuration 10C to the expanded configuration 10E, the urethral stent 10 expands in circumference and/or diameter, and a longitudinal length of the urethral stent 10 also expands.

As used herein, the “compressed configuration” is understood to mean the urethral stent 10 is compressed or unexpanded. Conversely, as used herein, the “expanded configuration” is understood to mean the urethral stent 10 has expanded outwardly from the compressed configuration 10C such that the urethral stent 10 has an increased circumference and/or an increased diameter relative to the compressed configuration 10C.

FIG. 1B is a simplified schematic side view illustration of the urethral stent 10 illustrated in FIG. 1A, the urethral stent 10 being shown in the compressed configuration 10C. Additionally, FIG. 1C is another simplified schematic side view illustration of the urethral stent 10 illustrated in FIG. 1A, the urethral stent now being shown in the expanded configuration 10E. FIG. 1B and FIG. 1C further illustrate various features and attributes that are incorporated into various embodiments of the urethral stent 10.

It is appreciated that a compressed diameter of the urethral stent 10 when in the compressed configuration 10C (as shown in FIG. 1B) should be small enough that the urethral stent 10 can be relatively easily moved into the urethra 611U (illustrated in FIG. 6) of the patient 611 (illustrated in FIG. 6) through use of the stent deployment system 220 (illustrated in FIG. 2). It is further appreciated that an expanded diameter of the urethral stent 10 when in the expanded configuration 10E (as shown in FIG. 1C) should be large enough that the urethral stent 10 can be firmly held and/or anchored in the desired location within the urethra 611U of the patient 611.

As illustrated, each of the first stent end 12 and the second stent end 14 have a flared design relative to a substantial majority of the stent body 16. More particularly, each of the first stent end 12 and the second stent end 14 comprises a flared end, such that a first end diameter 12D (illustrated in FIG. 1C) of the first stent end 12 (when in the expanded configuration 10E) and a second end diameter 14D (illustrated in FIG. 1C) of the second stent end 14 (when in the expanded configuration 10E) are larger than a body diameter 16D (illustrated in FIG. 1C) of the stent body 16 (when in the expanded configuration 10E). As utilized herein, the body diameter 16D of the stent body 16 is defined along a majority of the length of the stent body 16, other than the portions of the stent body 16 substantially immediately adjacent to the stent ends 12, 14 and/or as defined with bumps 18 along the length of the stent body 16. It is appreciated that in most embodiments the first end diameter 12D of the first stent end 12 will be substantially similar (approximately equal) to the second end diameter 14D of the second stent end 14. However, depending on the particular anatomical size (diameter) and shape of the urethra 611U into which the urethral stent 10 is being deployed, the urethral stent 10 can be designed so that the first end diameter 12D of the first stent end 12 and the second end diameter 14D of the second stent end 14 are different from one another. As described herein, the flared ends 12, 14 of the urethral stent 10 are usable to position and/or lock the urethral stent 10 in position relative to a wall of the urethra 611U, at a desired (preferred or optimal) location, such as close to the prostate and below the bladder neck, including at specified distances between the bladder neck opening and external urinary sphincter. For example, in certain implementations, the desired location for the urethral stent 10 can be within the urethra 611U and between approximately three and five millimeters from the bladder neck.

In various embodiments, a ratio of the first end diameter 12D of the first stent end 12 relative to the body diameter 16D of the stent body 16 (when in the expanded configuration 10E) can be between approximately 1.01:1 to 2.00:1. In some embodiments, the ratio of the first end diameter 12D of the first stent end 12 relative to the body diameter 16D of the stent body 16 (when in the expanded configuration 10E) can be between approximately 1.05:1 and 1.50:1. In other embodiments, the ratio of the first end diameter 12D of the first stent end 12 relative to the body diameter 16D of the stent body 16 (when in the expanded configuration 10E) can be between approximately 1.10:1 and 1.40:1. As such, in certain, non-exclusive alternative embodiments, the ratio of the first end diameter 12D of the first stent end 12 relative to the body diameter 16D of the stent body 16 (when in the expanded configuration 10E) can be approximately 1.01:1, 1.02:1, 1.03:1, 1.04:1, 1.05:1, 1.06:1, 1.07:1, 1.08:1, 1.09:1, 1.10:1, 1.15:1, 1.20:1, 1.25:1, 1.30:1, 1.35:1, 1.40:1, 1.45:1, 1.50:1, 1.55:1, 1.60:1, 1.65:1, 1.70:1, 1.75:1, 1.80:1, 1.85:1, 1.90:1, 1.95:1, or 2.00:1. Alternatively, the ratio of the first end diameter 12D of the first stent end 12 relative to the body diameter 16D of the stent body 16 (when in the expanded configuration 10E) can be different than the specific examples noted above.

Similarly, in various embodiments, a ratio of the second end diameter 14D of the second stent end 14 relative to the body diameter 16D of the stent body 16 (when in the expanded configuration 10E) can be between approximately 1.01:1 to 2.00:1. In some embodiments, the ratio of the second end diameter 14D of the second stent end 14 relative to the body diameter 16D of the stent body 16 (when in the expanded configuration 10E) can be between approximately 1.05:1 and 1.50:1. In other embodiments, the ratio of the second end diameter 14D of the second stent end 14 relative to the body diameter 16D of the stent body 16 (when in the expanded configuration 10E) can be between approximately 1.10:1 and 1.40:1. As such, in certain, non-exclusive alternative embodiments, the ratio of the second end diameter 14D of the second stent end 14 relative to the body diameter 16D of the stent body 16 (when in the expanded configuration 10E) can be approximately 1.01:1, 1.02:1, 1.03:1, 1.04:1, 1.05:1, 1.06:1, 1.07:1, 1.08:1, 1.09:1, 1.10:1, 1.15:1, 1.20:1, 1.25:1, 1.30:1, 1.35:1, 1.40:1, 1.45:1, 1.50:1, 1.55:1, 1.60:1, 1.65:1, 1.70:1, 1.75:1, 1.80:1, 1.85:1, 1.90:1, 1.95:1, or 2.00:1. Alternatively, the ratio of the second end diameter 14D of the second stent end 14 relative to the body diameter 16D of the stent body 16 (when in the expanded configuration 10E) can be different than the specific examples noted above.

As further illustrated, in many embodiments, the stent body 16 can include one or more bumps 18 (also referable to as waves, undulations or ribs) along a length of the stent body 16 from the first stent end 12 to the second stent end 14. For example, in some embodiments, the stent body 16 can include between one and ten bumps 18 along the length of the stent body 16. More particularly, in certain embodiments, the stent body 16 can include one, two, three, four, five, six, seven, eight, nine, or ten bumps 18 along the length of the stent body 16. Alternatively, the stent body 16 can include more than ten bumps 18 along the length of the stent body 16. Still alternatively, the stent body 16 can be designed without any bumps 18 along the length of the stent body 16.

In many implementations, as described in greater detail herein below, the bumps 18 that are included along the length of the stent body 16 are provided to help lock the urethral stent 10 to various sections of the urethra 611U. More particularly, the bumps 18 can inhibit the urethral stent 10 from sliding or moving along a length of the urethra 611U during times of contraction and extension of the penis. It is appreciated that the size of the bumps 18 can be varied as deemed necessary in order to effectively lock the urethral stent 10 in a desired position such that the urethral stent 10 is inhibited from sliding or moving along a length of the urethra 611U during times of contraction and extension of the penis. More specifically, it is appreciated that the size of the bumps 18 can be different (larger or smaller) than what is illustrated in the Figures.

As shown, in certain embodiments, the bumps 18 can be designed to provide a consistent, repeating pattern along a full length of the stent body 16. Alternatively, the bumps 18 can provide an inconsistent pattern along the full length of the stent body 16.

As further illustrated, in many embodiments, the bumps 18 are configured to extend outwardly about a full circumference of the stent body 16. Alternatively, in other embodiments, the bumps 18 can be configured to extend outwardly from the stent body 16 about less than the full circumference of the stent body 16. For example, in one non-exclusive alternative embodiment, the bumps 18 can be staggered relative to one another such that the bumps 18 only extend outwardly from a portion of the circumference of the stent body 16, which alternates from one side of the stent body 16 to the other along the length of the stent body 16.

It is appreciated that the bumps 18 can be formed into the stent body 16 in any suitable manner. For example, in certain non-exclusive implementations, the bumps 18 can be formed into a straight, generally cylindrical stent body 16 through use of a specially designed tool, which includes bumps (raised diameter) at various locations, that is configured to push the body diameter 16D of the stent body 16 outwardly at various locations along the length and/or about the circumference of the stent body 16. In one implementation, this can be accomplished by annealing the urethral stent 10 inside a furnace. Alternatively, the bumps 18 can be formed into the stent body 16 in another suitable manner.

The urethral stent 10 can be formed from any suitable materials. For example, in some embodiments, the urethral stent 10 can be formed from one or more of shape memory materials, alloys, spring materials, and super elastic materials including Nitinol (nickel-titanium alloy), Nitinol-based alloys, cobalt chromium alloys, spring steels, and spring stainless steels. In other embodiments, the urethral stent 10 can be formed from one or more of any other known shape memory materials including poly-ether-ether-ketone (PEEK), and shape memory and bio-absorbable polymers and metals (such as polylactic acid, polyglycolic acid and their copolymers, and magnesium alloys). In still other embodiments, the urethral stent 10 can be formed from other suitable materials.

As an overview, the present invention is directed toward devices and methods of treatment and device manufacturing to provide a urethral stent 10 and associated stent deployment system 220 (illustrated in FIG. 2) for the treatment of various BPH symptoms, such as urinary outflow obstruction symptoms. In various embodiments, the urethral stent 10 has a three-dimensional bumped, ribbed, or wavy surface and flared ends 12, 14 that are used to position and lock the urethral stent 10 within a vessel wall or urethral wall close to the prostate and below the bladder neck. The bumps 18 can further assist with anchoring the urethral stent 10 at various points along the length of the urethral stent 10. In many embodiments, the design and implementation of the stent deployment system 220 enables the urethral stent 10 to be effectively and accurately deployed to a desired location within the urethra 611U of the patient 611.

FIG. 2 is a simplified schematic side view illustration of an embodiment of the urethral stent 210, and an embodiment of a stent deployment system 220 having features of the present invention that is usable for positioning the urethral stent 210 within a urethra 611U (illustrated in FIG. 6) of a patient 611 (illustrated in FIG. 6). As described herein, the urethral stent 210 and the stent deployment system 220 can be referred to collectively as a stent assembly 222.

As illustrated in FIG. 2, the urethral stent 210 can be substantially identical in design and function to the urethral stent 10 illustrated and described above in relation to FIGS. 1A-1C. For example, the urethral stent 210 can again include a flared, first stent end 212, a flared, second stent end 214, and a stent body 216 with one or more bumps 218, which are substantially identical in design and function to the corresponding components of the urethral stent 10 illustrated and described above in relation to FIGS. 1A-1C. Accordingly, the urethral stent 210 will not be described again in substantial detail.

The stent deployment system 220 is configured to effectively deploy and/or position the urethral stent 210 within the urethra 611U of the patient 611. More particularly, as illustrated and described, the urethral stent 210 is effectively and accurately deployed through use of the stent deployment system 220 to a desired location within the urethra 611U of the patient 611.

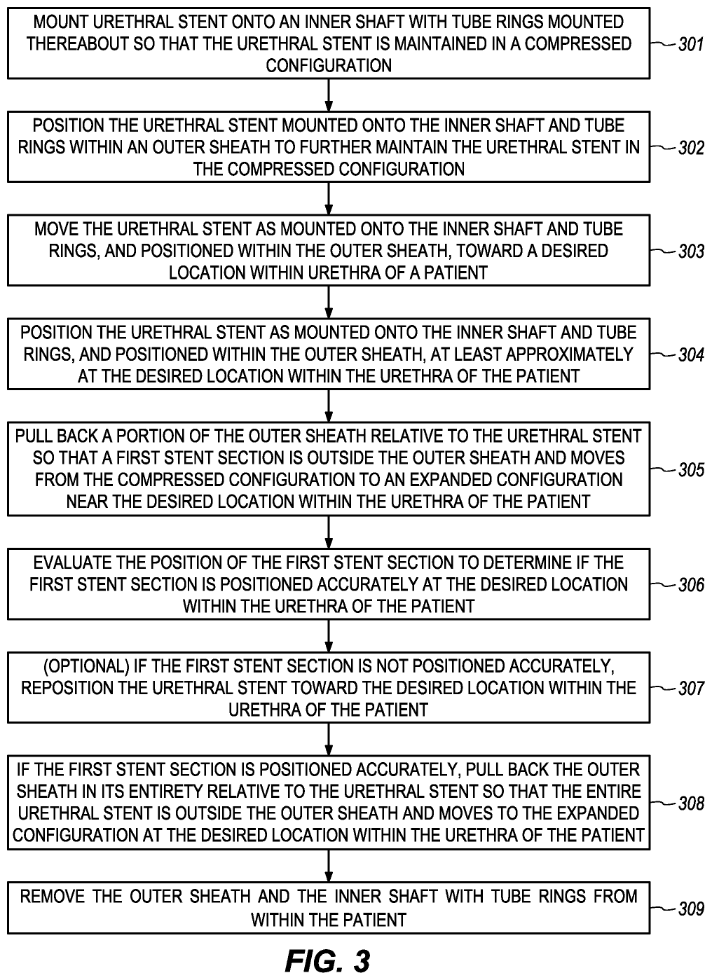

The design of the stent deployment system 220 can be varied to suit the requirements of the urethral stent 210 and the patient 611 within which the urethral stent 210 is to be deployed. In various embodiments, as shown in FIG. 2, the stent deployment system 220 can include one or more of an inner shaft 224, at least one tube ring 226 (four tube rings 226 are specifically illustrated in FIG. 2) that is mounted about the inner shaft 224, and an outer sheath 228. Alternatively, the stent deployment system 220 can include more components or fewer components than those which are specifically illustrated in FIG. 2. For example, in some alternative embodiments, the stent deployment system 220 can further include and/or utilize a crimping head 430 (illustrated in FIG. 4) for initially crimping the urethral stent 210 onto the inner shaft 224 and/or the at least one tube ring 226 that is mounted about the inner shaft 224. By crimping the urethral stent 210 onto the inner shaft 224 and/or the tube ring(s) 226, the urethral stent 210 can be effectively maintained in the compressed configuration 10C (illustrated in FIG. 1B) prior to being positioned within the outer sheath 228 and prior to being deployed within the urethra 611U of the patient 611.

In various embodiments, the inner shaft 224 is a generally cylindrical-shaped shaft that can be formed from any suitable rigid material. The inner shaft 224 is configured to provide a suitable rigid surface about which the urethral stent 210 can be mounted and/or crimped so that the urethral stent 210 can be maintained in the compressed configuration 10C. Alternatively, the inner shaft 224 can have another suitable design and/or can be formed from other suitable materials.

The inner shaft 224 can have any suitable length, although it is appreciated that the inner shaft 224 will typically have a length that is at least as great as a length of the urethral stent 210. With such design, the urethral stent 210 will have a surface on which to be positioned about along a full length of the urethral stent 210.

The tube ring(s) 226 are spaced apart from one another and are mounted about the inner shaft 224. In some embodiments, the tube rings 226 are substantially evenly spaced apart from one another along the length of the inner shaft 224, and help to define a plurality of stent sections 232 (defined by dotted lines) of the urethral stent 210 when the urethral stent 210 is mounted about the inner shaft 224 and/or the tube rings 226. For example, in certain implementations, when the urethral stent 210 is mounted about the inner shaft 224 and/or the tube rings 226, each section 232 of the urethral stent 210 can substantially coincide with a single tube ring 226. Additionally, or in the alternative, in some implementations, the tube rings 226 as mounted about the inner shaft 224, and/or the sections 232 of the urethral stent 210, can substantially coincide with the bumps 218 included within the stent body 216. Still alternatively, the tube rings 226 can be unevenly spaced apart from one another along the length of the inner shaft 224.

As described in greater detail herein below, the tube ring(s) 226 enable the urethral stent 210 to be accurately positioned and/or repositioned during deployment of the urethral stent 210 at the desired location within the urethra 611U of the patient 611. More particularly, the tube ring(s) 226 allow a user or operator to move the urethral stent 210 out of or back into the outer sheath 228 during positioning and/or repositioning of the urethral stent 210 at the desired location. In many implementations, the urethral stent 210 is deployed (positioned and/or repositioned) at the desired location within the urethra 611U of the patient 611 on a section-by-section basis, with each of the stent sections 232 being deployed one after the other, and with the desired location being confirmed as each stent section 232 is individually deployed. For purposes of repositioning the urethral stent 210 as deemed necessary, in instances where it is determined that the urethral stent 210 is positioned so as to extend in an undesired manner, such as inside the bladder neck, the user or operator can easily pull the urethral stent 210 back into the outer sheath 228 because the urethral stent 210 is still crimped onto one or more tube rings 226 that are still within the outer sheath 228. Such repositioning of the urethral stent 210 is critical as it helps to achieve optimal urine flow without risk of the urethral stent 210 falling inside the bladder neck.

In certain embodiments, the tube ring(s) 226 can have an outer diameter that is approximately equal to an inner diameter of the outer sheath 228, less a thickness of the stent body 216 of the urethral stent 210, which, as described in detail herein below, can be positioned between the inner shaft 224, with the tube ring(s) 226 mounted thereabout, and the outer sheath 228 during initial deployment of the urethral stent 210 at the desired location within the urethra 611U of the patient 611.

The stent deployment system 220 can include any suitable number of tube rings 226 (four tube rings 226 are illustrated in FIG. 2) that are mounted about the inner shaft 224. In many embodiments, the number of tube rings 226 can be dependent on a longitudinal length of the urethral stent 10. In certain embodiments, the stent deployment system 220 can include from one to ten tube rings 226. More particularly, in such embodiments, the stent deployment system 220 can include one, two, three, four, five, six, seven, eight, nine, or ten tube rings 226. In other embodiments, the stent deployment system 220 can include from two to seven tube rings 226. In still other embodiments, the stent deployment system 220 can include three or four tube rings 226. Alternatively, the stent deployment system 220 can include greater than ten tube rings 226 or no tube rings 226.

In various embodiments, the stent deployment system 220 includes at least two tube rings 226 that are mounted about the inner shaft 224, such that when the urethral stent 210 is crimped onto the inner shaft 224 and/or the tube rings 226, one tube ring 226 is positioned near the first stent end 212 (adjacent to the flared section) and one tube ring 226 is positioned near the second stent end 214 (adjacent to the flared section). Depending on the length of the urethral stent 210 and the inner shaft 224, any additional number of tube rings 226 can be further mounted about the inner shaft 224 between the tube rings 226 that are positioned near the first stent end 212 and the second stent end 214.

The tube ring(s) 226 can be formed from any suitable material. For example, in certain non-exclusive embodiments, the tube ring(s) are formed from a soft durometer material. Alternatively, the tube ring(s) 226 can be formed from another suitable resilient material.

The outer sheath 228 is substantially cylinder-shaped, and is sized such that the urethral stent 210, as mounted about the inner shaft 224 and/or the tube rings 226, can fit therewithin when in the compressed configuration 10C. In particular, in various embodiments, the outer sheath 228 will include an inner diameter that is large enough so that the urethral stent 210, as mounted about the inner shaft 224 and/or the tube rings 226, can be positioned therewithin when in the compressed configuration 10C. Stated in another manner, during use of the stent deployment system 220 to deploy the urethral stent 210 within the urethra 611U of the patient 611, the outer sheath 228 is positioned to substantially encircle the inner shaft 224 and the tube rings 226 with the urethral stent 210 mounted thereabout.

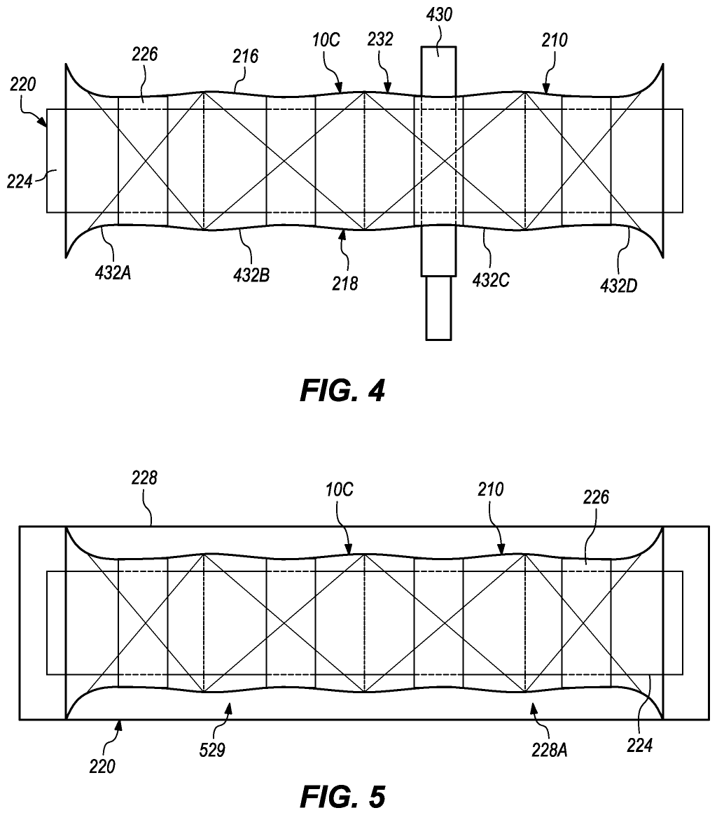

In many embodiments, the inner diameter of the outer sheath 228 can be greater than an outer diameter of the inner shaft 224 and the tube rings 226, such that the outer sheath 228 is spaced apart from the inner shaft 224 and the tube rings 226 to define a system gap 529 (illustrated in FIG. 5) therebetween. During initial deployment of the urethral stent 210, as the urethral stent 210 is mounted about the inner shaft 224 and the tube rings 226, the urethral stent 210 can be selectively positioned within the system gap 529.

Moreover, the outer sheath 228 can define an inner cavity 228A therein, and the urethral stent 210 when in the compressed configuration 10C as mounted about the inner shaft 224 and/or the tube rings 226 can be positioned substantially fully within the inner cavity 228A as defined by the outer sheath 228. As described, during initial positioning and/or deployment within the urethra 611U of the patient 611, the urethral stent 210 is typically positioned substantially fully within the outer sheath 228 so that the urethral stent 210 is further maintained in the compressed configuration 10C.

In many embodiments, the outer sheath 228 can have an outer diameter that fits within and can be moved relative to the urethra 611U of the patient 611. More specifically, the outer diameter of the outer sheath 228 will typically be at least slightly smaller than an inner diameter of the urethra 611U of the patient 611 so as to be easily movable and positionable within the urethra 611U of the patient 611 during initial positioning and/or deployment of the urethral stent 210 with the urethral stent 210 in the compressed configuration 10C.

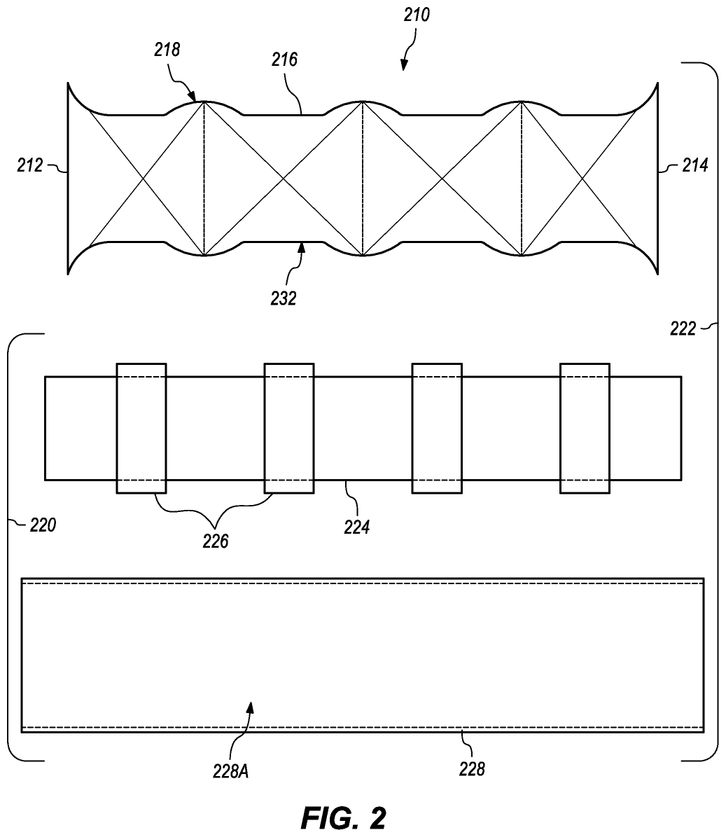

FIG. 3 is a flowchart that illustrates a method for utilizing the stent deployment system of FIG. 2 to position the urethral stent at a desired location within the urethra of the patient. It is recognized that in nonexclusive alternative embodiments, the system and method of FIG. 3 can include additional steps other than those specifically delineated herein or can omit certain of the steps that are specifically delineated herein. Moreover, in some embodiments, the order of the steps described below can be modified and/or certain steps can be combined without deviating from the spirit of the present invention.

At step 301, a urethral stent, having features of the present invention, is crimped and/or mounted onto an inner shaft so that the urethral stent is maintained in a compressed configuration. In some embodiments, a crimping head is specifically utilized to crimp and/or mount the urethral stent onto the inner shaft. In many embodiments, the inner shaft can have at least one tube ring mounted thereabout. In such embodiments, the urethral stent will be crimped and/or mounted about the inner shaft and/or the tube ring(s). In embodiments that include more than one tube ring, the tubes rings will be spaced apart from one another. As described in greater detail herein below, the tube rings are provided to better enable the positioning and potential repositioning of the urethral stent at the desired location within the urethra of the patient.

Referring now to FIG. 4, FIG. 4 is a simplified schematic side view illustration of the urethral stent 210 being mounted (crimped) onto a portion of the stent deployment system 220 so that the urethral stent 210 is in the compressed configuration 10C. More particularly, as shown, a crimping head 430 is being utilized so that the urethral stent 210 is mounted (crimped) onto the inner shaft 224 and/or the tube rings 226 of the stent deployment system 220. During the crimping process, the urethral stent 210 is initially placed over the inner shaft 224 and/or the tube rings 226, and then is placed inside the crimping head 430. In certain implementations, the crimping head 430 then collapses to roughly half the size of the inner diameter of the outer shaft 228 (illustrated in FIG. 2) so that the crimped urethral stent 210 can be positioned correctly. Then, the crimping head 430 is utilized to compress the urethral stent 210 down even further, such as approximately 0.01 inches to 0.03 inches further down, so that the urethral stent 210, as crimped onto the inner shaft 224 and/or the tube rings 226, can be pulled out from the crimping head 430 and positioned within the outer sheath 228.

In certain embodiments, as shown, the bumps 218 of the stent body 216 can coincide with the tube rings 226 that are mounted about the inner shaft 224. Moreover, the bumps 218 of the stent body 216 and/or the tube rings 226 mounted about the inner shaft 224 can be utilized to define a plurality of stent sections 232 (defined by dotted lines) of the urethral stent 210. For example, in the embodiment illustrated in FIG. 4, the bumps 218 of the stent body 216 and/or the tube rings 226 mounted about the inner shaft 224 are utilized to define four sections 232 of the urethral stent 210, i.e. a first stent section 432A, a second stent section 432B, a third stent section 432C, and a fourth stent section 432D. As described in greater detail herein below, during deployment of the urethral stent 210 within the urethra 611U (illustrated in FIG. 6) of the patient 611 (illustrated in FIG. 6), the urethral stent 210 is positioned and/or deployed section-by-section at the desired location within the urethra 611U of the patient 611.

Returning to FIG. 3, at step 302, the urethral stent that is mounted (crimped) on the inner shaft and/or the tube rings is positioned within an outer sheath. More particularly, after the urethral stent is mounted (crimped) about the inner shaft and/or the tube rings, the outer sheath can be inserted close to the end of the crimping head and the inner shaft and tube rings with the urethral stent crimped thereabout. The urethral stent as mounted/crimped about the inner shaft and the tube rings is then pulled inside the outer sheath until the entirety of the crimped, urethral stent is positioned within the outer sheath at a predetermined distance. It is appreciated that the urethral stent has to be mounted and/or crimped onto the inner shaft and/or the tube rings, and within the outer sheath, in order that the urethral stent can be effectively maintained in the compressed configuration before the actual deployment of the urethral stent.

Referring now to FIG. 5, FIG. 5 is a simplified schematic side view illustration of the urethral stent 210 that has been mounted and/or crimped about the inner shaft 224 and/or the tube rings 226 now being positioned within the outer sheath 228 of the stent deployment system 220 so that the urethral stent 210 remains in the compressed configuration 10C. More particularly, as shown, the urethral stent 210 as mounted about the inner shaft 224 and/or the tube rings 226 is now at least substantially fully positioned within an inner cavity 228A as defined by the outer sheath 228 such that the urethral stent 210 is maintained in the compressed configuration 10C.

Returning again to FIG. 3, at step 303, the urethral stent, as mounted onto the inner shaft and/or the tube rings, and as positioned within the outer sheath, is moved toward a desired location within a urethra of a patient.

Referring now to FIG. 6, FIG. 6 is a simplified schematic side view illustration of the urethral stent 210 and the stent deployment system 220 of FIG. 2 during initial deployment of the urethral stent 210 toward the desired location within the urethra 611U of the patient 611. As shown, during such initial deployment of the urethral stent 210, the urethral stent 210 is still mounted and/or crimped about the inner shaft 224 and/or the tube rings 226, and is still positioned at least substantially fully, if not completely, within the inner cavity 228A of the outer sheath 228.

Returning to FIG. 3, at step 304, the urethral stent, as mounted onto the inner shaft and/or the tube rings, and as positioned within the outer sheath, is positioned at least approximately at the desired location within the urethra of the patient. As described herein, in various implementations, the desired location for the urethral stent is within the urethra and adjacent to the urethral wall, proximate to the prostate and below the bladder neck, including at specified distances between the bladder neck opening and the external urinary sphincter.

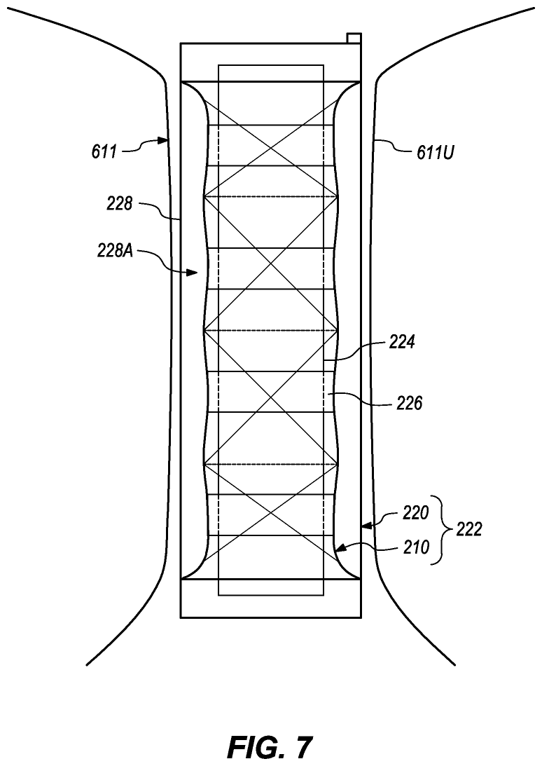

Referring now to FIG. 7, FIG. 7 is a simplified schematic side view illustration of the urethral stent 210 and the stent deployment system 220 of FIG. 2 as the urethral stent 210 and the stent deployment system 220, also referred to collectively as the stent assembly 222, have arrived at least approximately at the desired location within the urethra 611U of the patient 611. As shown, when the urethral stent 210, the stent deployment system 220 and/or the stent assembly 222 initially arrive at least approximately at the desired location within the urethra 611U of the patient 611, the urethral stent 210 is still mounted and/or crimped about the inner shaft 224 and the tube rings 226, and is still positioned substantially fully within the inner cavity 228A as defined by the outer sheath 228 of the stent deployment system 220.

Returning again to FIG. 3, at step 305, a portion of the outer sheath is pulled back or withdrawn relative to the urethral stent so that a first stent section of the urethral stent is no longer positioned within the outer sheath. It is appreciated that at some point prior to and/or while the outer sheath is being pulled back or withdrawn relative to the urethral stent, and the first stent section moving outside the outer sheath, the crimping of the first stent section onto the inner shaft and/or the tube rings is removed or disengaged. In particular, in certain implementations, during the initial deployment process, the inner shaft can be pushed from a proximal end relative to the outer sheath so that the urethral stent can be slowly deployed outside the outer sheath.

As now positioned outside the outer sheath, and no longer being crimped onto the inner shaft and/or the tube rings, the first stent section of the urethral stent moves from the compressed configuration to an expanded configuration, and can be effectively deployed at or near the desired location within the urethra of the patient. Stated in another manner, once the outer sheath is pulled back or withdrawn relative to the urethral stent, the urethral stent can be deployed to the desired location by releasing the first stent section (and then other stent sections of the urethral stent one by one) to make sure that the urethral stent is accurately deployed at the desired location.

In some embodiments, as noted above, the urethral stent has a self-expanding stent design such that when the first stent section moves outside the outer sheath, and with the crimping removed and/or disengaged, the first stent section will spontaneously move from the compressed configuration to the expanded configuration. Alternatively, the first stent section can be moved from the compressed configuration to the expanded configuration in another suitable manner.

In certain implementations, the pulling back or withdrawing of the outer sheath relative to the urethral stent can be accomplished by having separate and/or independent control of the positioning and movement of the outer sheath and the inner shaft. In particular, as the outer sheath is being pulled back or withdrawn by the user or operator, the inner shaft (with the tube rings mounted thereabout) can be effectively held steady at its previous location (such as by providing a slight pushing force on the inner shaft relative to the outer sheath). Since a portion of the urethral stent is still mounted and/or crimped onto the inner shaft and/or the tube rings (such as the remaining sections of the urethral stent other than the first stent section), the positioning of the urethral stent will coincide with the positioning of the inner shaft. Thus, by effectively holding the inner shaft steady at the desired location, the urethral stent will also remain at the desired location while the outer sheath is pulled back or withdrawn. It is appreciated that although the inner shaft (and thus the urethral stent) and the outer sheath are initially positioned within the urethra of the patient together, with the inner shaft and the urethral stent positioned within the inner cavity of the outer sheath, the means of controlling the movement of the inner shaft and the outer sheath can still be independent of one another.

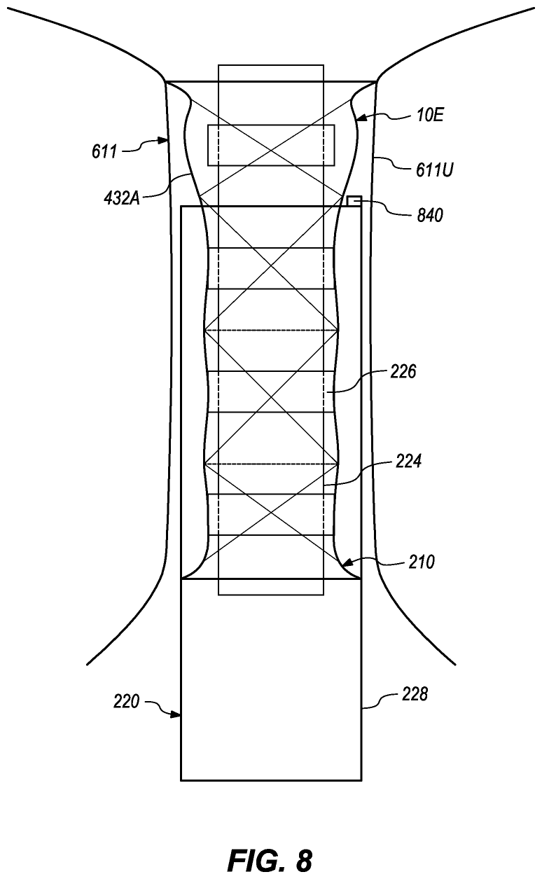

Referring now to FIG. 8, FIG. 8 is a simplified schematic side view illustration of the urethral stent 210 and the stent deployment system 220 of FIG. 2 after partial withdrawal of the outer sheath 228 relative to the urethral stent 210, with the urethral stent 210 now having moved partially toward the expanded configuration 10E and being partially positioned at least approximately at the desired location within the urethra 611U of the patient 611. In particular, as shown, the first stent section 432A of the urethral stent 210 is now positioned outside the outer sheath 228. Since the crimping of the first stent section 432A onto the inner shaft 224 and/or the tube rings 226 has also been removed or disengaged, the first stent section 432A has now also been allowed to move from the compressed configuration 10C (such as illustrated in FIG. 4) to the expanded configuration 10E.

As noted, it is desired to provide a means by which accurate deployment of the urethral stent 210 at the desired location within the urethra 611U of the patient 611 can be confirmed. For example, in some embodiments, the outer sheath 228 can have an imaging system 840, such as a CMOS image sensor in certain non-exclusive embodiments, coupled thereto, which can be utilized to determine if the first stent section 432A of the urethral stent 210 is accurately positioned at the desired location within the urethra 611U of the patient 611. More particularly, the imaging system 840 enables the user or operator to visualize the urethral stent 210 (including the first stent section 432A) and see if the urethral stent 210 (and more specifically the first stent section 432A at this point) is accurately positioned as desired within the urethra 611U of the patient 611. It is appreciated that having the imaging system 840, such as the CMOS image sensor, positioned at a tip of the outer sheath 228 allows this procedure to be conducted within a clinic rather than requiring a hospital setting. This can be preferred, especially in areas where access to suitable hospital facilities may be more limited.

Returning once again to FIG. 3, at step 306, the positioning of the first stent section of the urethral stent, now in the expanded configuration, is evaluated to determine if the first stent section of the urethral stent is positioned properly and accurately at the desired location within the urethra of the patient.

As described, the methods of the present invention include orienting a distal tip of the stent deployment system within the urethra and adjacent to the prostate, and incrementally section-by-section deploying the urethral stent from the compressed configuration to the expanded configuration. Stated in another manner, the evaluation of the proper positioning of the urethral stent at the desired location within the urethra of the patient can be undertaken as any and all stent sections are individually released from within the outer sheath and move from the compressed configuration to the expanded configuration. With such design, it is appreciated that that deployment of the urethral stent may be interrupted as deemed necessary and/or appropriate between expansion of the urethral stent from the compressed configuration to the expanded configuration in order to reorient or relocate the urethral stent along the length of the urethra and within the prostate.

At optional step 307, if the evaluation of step 306 determines that the urethral stent is not positioned properly at the desired location within the urethra of the patient, the position of the urethral stent is adjusted toward the desired location. More particularly, in order to adjust the position of the urethral stent, the inner shaft is first moved fully back within the outer sheath before the positioning of the stent assembly as a whole is repositioned or reoriented in an appropriate manner relative to the true desired location within the urethra of the patient. To move the inner shaft, and thus the entirety of the urethral stent, fully back within the outer sheath can again entail a coordinated push-and-pull action between the outer sheath and the inner shaft. For example, the movement and positioning of the outer sheath and the inner shaft can again be controlled separately and/or independently of one another. Thus, by pushing the outer sheath back further into the urethra while pulling back slightly on the inner shaft, the inner shaft, and thus the entirety of the urethral stent, can then again be positioned fully within the inner cavity of the outer sheath. Once the inner shaft and urethral stent are again positioned fully within the inner cavity of the outer sheath, the position of the stent assembly as a whole can be adjusted as necessary so that the stent assembly is more precisely and properly positioned at the desired location within the urethra of the patient. It is appreciated that the repositioning of the stent assembly within the urethra of the patient will be based, at least in part, on the information gathered in evaluation step 306 as to the actual positioning of the first stent section of the urethral stent within the urethra of the patient.

It is further appreciated that this optional step 307 effectively returns the procedure back to step 304 as described above, so that the stent assembly can be more accurately and precisely positioned within the urethra of the patient.

At step 308, if the evaluation of step 306 determines that the urethral stent is positioned properly at the desired location within the urethra of the patient, then the outer sheath can be pulled back or withdrawn in its entirety relative to the urethral stent (stent section by stent section) so that the entire urethral stent is no longer positioned within the outer sheath. It is appreciated that at some point prior to the outer sheath being pulled back or withdrawn in its entirety relative to the urethral stent, and the entire urethral stent moving outside the outer sheath, the crimping of each stent section of the urethral stent onto the inner shaft and/or the tube rings is fully removed or disengaged.

As now positioned fully outside the outer sheath, and no longer being crimped onto the inner shaft and/or the tube rings, the urethral stent in its entirety moves from the compressed configuration to the expanded configuration. At this point, the urethral stent is now fully positioned in the expanded configuration at the desired location within the urethra of the patient.

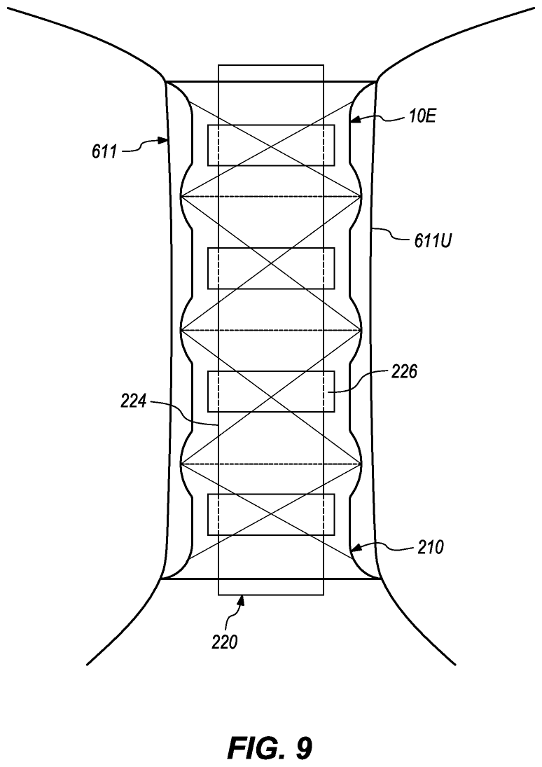

Referring now to FIG. 9, FIG. 9 is a simplified schematic side view illustration of the urethral stent 210 and the stent deployment system 220 of FIG. 2 after full withdrawal of the outer sheath 228 (illustrated in FIG. 2) relative to the urethral stent 210, with the urethral stent 210 now fully in the expanded configuration 10E and being fixed in position at the desired location within the urethra 611U of the patient 611. In particular, in FIG. 9, the outer sheath 228 is no longer shown. However, the inner shaft 224 and/or the tube rings 226 are still illustrated, although the urethral stent 210 is no longer mounted and/or crimped onto the inner shaft 224 and/or the tube rings 226.

As described, the methods of the present invention include the design of the unique urethral stent 210 having a three-dimensional bumped, ribbed, or wavy surface and flared ends that once it is deployed, the urethral stent 210 can anchor firmly at both ends of the vessel or urethral walls such as close to the prostate and below the bladder neck wall, including at specified distances between the bladder neck opening and external urinary sphincter. The bumps 218 can further assist with anchoring the urethral stent 210 at various points along the length of the urethral stent 210.

Returning yet again to FIG. 3, at step 309, the outer sheath and the inner shaft are removed from within the patient. The urethral stent is now positioned for long-term deployment within the urethra of the patient.

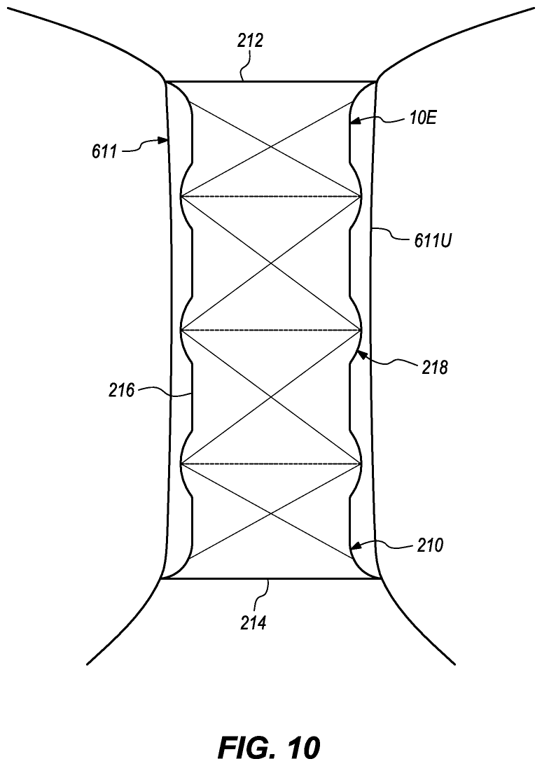

Referring now to FIG. 10, FIG. 10 is a simplified schematic side view illustration of the urethral stent 210 after full withdrawal of the stent deployment system 220 (illustrated in FIG. 2) relative to the urethral stent 210, with the urethral stent 210 now fully in the expanded configuration 10E and being fixed in position at the desired location within the urethra 611U of the patient 611. More particularly, the unique design of the urethral stent 210, including the flared stent ends 212, 214 and the stent body 216 including one or more bumps 218, better enable the urethral stent 210 to be locked and/or anchored in position within the urethra 611U of the patient 611, close to the prostate and below the bladder neck. Additionally, as noted above, the bumps 218 that are included along the length of the stent body 216 further help to lock the urethral stent 210 to various sections of the urethra 611U. More particularly, the bumps 218 can inhibit the urethral stent 210 from sliding or moving along a length of the urethra 611U during times of contraction and extension of the penis. With such design, the anchoring of the urethral stent 210 within the urethra 611U of the patient 611 can occur not only at the flared stent ends 212, 214, but also at each of the bumps 218 having the largest diameter. It is appreciated that the size of the bumps 218 can be varied as deemed necessary in order to effectively lock the urethral stent 210 in a desired position such that the urethral stent 210 is inhibited from sliding or moving along a length of the urethra 611U during times of contraction and extension of the penis.

It is understood that although a number of different embodiments of the urethral stent 10, the stent deployment system 220, and/or the stent assembly 222 have been illustrated and described herein, one or more features of any one embodiment can be combined with one or more features of one or more of the other embodiments, provided that such combination satisfies the intent of the present invention.

While a number of exemplary aspects and embodiments of the urethral stent 10, the stent deployment system 220, and/or the stent assembly 222 have been discussed above, those of skill in the art will recognize certain modifications, permutations, additions, and sub-combinations thereof. It is therefore intended that the following appended claims and claims hereafter introduced are interpreted to include all such modifications, permutations, additions, and sub-combinations as are within their true spirit and scope.

What is claimed is:

1. A stent that is configured to be positioned within a urethra of a patient, the stent comprising:
a first stent end;
an opposed second stent end; and
a stent body that extends between the first stent end and the second stent end;
wherein at least one of the first stent end and the second stent end is flared relative to the stent body.

2. The stent of claim 1 wherein each of the first stent end and the second stent end is flared relative to the stent body.

3. The stent of claim 1 wherein the first stent end has a first end diameter; wherein the stent body has a body diameter; and wherein a ratio of the first end diameter of the first stent end to the body diameter of the stent body is between approximately 1.05:1 and 1.50:1.

4. The stent of claim 3 wherein the second stent end has a second end diameter; and wherein a ratio of the second end diameter of the second stent end to the body diameter of the stent body is between approximately 1.05:1 and 1.50:1.

5. The stent of claim 1 wherein the stent body has one or more bumps along the stent body from the first stent end to the second stent end.

6. The stent of claim 5 wherein the stent body has a plurality of bumps along the stent body from the first stent end to the second stent end.

7. The stent of claim 1 wherein the stent body is substantially tubular-shaped; and wherein the stent is configured to move from a compressed configuration as the stent is initially positioned within the urethra of the patient, to an expanded configuration as the stent is deployed at a desired location within the urethra of the patient.

8. The stent of claim 7 wherein the stent is a self-expanding stent that is biased to spontaneously move from the compressed configuration to the expanded configuration.

9. A stent assembly comprising the stent of claim 1; and a stent deployment system that is configured to position the stent within the urethra of the patient.

10. The stent assembly of claim 9 wherein the stent deployment system includes an inner shaft, at least one tube ring positioned about the inner shaft, and an outer sheath that is configured to substantially encircle the inner shaft and the at least one tube ring, the outer sheath defining an inner cavity therewithin; wherein the stent is mounted about the inner shaft and the at least one tube ring prior to being positioned within the urethra of the patient so that the stent is maintained in a compressed configuration; and wherein the stent as mounted about the inner shaft and the at least one tube ring is positioned within the inner cavity of the outer sheath prior to being positioned within the urethra of the patient.

11. A stent assembly comprising:
a stent including a first stent end, an opposed second stent end, and a stent body that extends between the first stent end and the second stent end; and
a stent deployment system that is configured to position the stent within a urethra of a patient, the stent deployment system including an inner shaft, at least one tube ring positioned about the inner shaft, and an outer sheath that is configured to substantially encircle the inner shaft and the at least one tube ring, the outer sheath defining an inner cavity therewithin;
wherein the stent is positioned within the inner cavity of the outer sheath as the stent is being positioned within the urethra of the patient.

12. The stent assembly of claim 11 wherein the stent is mounted about the inner shaft and the at least one tube ring prior to being positioned within the urethra of the patient so that the stent is maintained in a compressed configuration.

13. The stent assembly of claim 12 further comprising a crimping head that is configured to crimp the stent onto the inner shaft and the at least one tube ring prior to being positioned within the urethra of the patient so that the stent is maintained in the compressed configuration.

14. The stent assembly of claim 12 wherein the stent as mounted about the inner shaft and the at least one tube ring is positioned within the inner cavity of the outer sheath prior to being positioned within the urethra of the patient.

15. The stent assembly of claim 14 wherein each of the first stent end and the second stent end is flared relative to the stent body.

16. The stent assembly of claim 15 wherein the first stent end has a first end diameter, the second stent end has a second end diameter, and the stent body has a body diameter; wherein a ratio of the first end diameter of the first stent end to the body diameter of the stent body is between approximately 1.05:1 and 1.50:1; and wherein a ratio of the second end diameter of the second stent end to the body diameter of the stent body is between approximately 1.05:1 and 1.50:1.

17. The stent assembly of claim 14 wherein the stent body has one or more bumps along the stent body from the first stent end to the second stent end.

18. The stent assembly of claim 14 wherein the stent body is substantially tubular-shaped; and wherein the stent is configured to move from the compressed configuration as the stent is initially positioned within the urethra of the patient, to an expanded configuration as the stent is deployed at a desired location within the urethra of the patient.

19. The stent assembly of claim 18 wherein the stent is a self-expanding stent that is biased to spontaneously move from the compressed configuration to the expanded configuration when the stent is removed from within the inner cavity of the outer sheath.

20. A stent assembly comprising:
a stent including a first stent end, an opposed second stent end, and a stent body that extends between the first stent end and the second stent end, the stent body having one or more bumps along the stent body from the first stent end to the second stent end, each of the first stent end and the second stent end being flared relative to the stent body; and
a stent deployment system that is configured to position the stent within a urethra of a patient, the stent deployment system including an inner shaft, at least one tube ring positioned about the inner shaft, and an outer sheath that is configured to substantially encircle the inner shaft and the at least one tube ring, the outer sheath defining an inner cavity therewithin;
wherein the stent is mounted about the inner shaft and the at least one tube ring prior to being positioned within the urethra of the patient so that the stent is maintained in a compressed configuration;
wherein the stent as mounted about the inner shaft and the at least one tube ring is positioned within the inner cavity of the outer sheath prior to being positioned within the urethra of the patient so that the stent is further maintained in the compressed configuration;
wherein the first stent end has a first end diameter, the second stent end has a second end diameter, and the stent body has a body diameter;
wherein a ratio of the first end diameter of the first stent end to the body diameter of the stent body is between approximately 1.05:1 and 1.50:1;
wherein a ratio of the second end diameter of the second stent end to the body diameter of the stent body is between approximately 1.05:1 and 1.50:1;
wherein the stent is configured to move from the compressed configuration as the stent is initially positioned within the urethra of the patient, to an expanded configuration as the stent is deployed at a desired location within the urethra of the patient; and
wherein the stent is a self-expanding stent that is biased to spontaneously move from the compressed configuration to the expanded configuration when the stent is removed from within the inner cavity of the outer sheath.
